(19) United States
(12) Patent Application Publication
(10) Pub. No.: US 2022/0168727 A1
(43) Pub. Date: Jun. 2, 2022

(54) BIOSENSOR FOR DETECTION OF ANALYTES IN A FLUID

(71) Applicant: Mehul BALDWA, Indore (IN)

(72) Inventor: Mehul BALDWA, Indore (IN)

(21) Appl. No.: 17/442,336

(22) PCT Filed: Mar. 24, 2020
(86) PCT No.: PCT/IB2020/052736

§ 371 Date: Sep. 23, 2021

(30) Foreign Application Priority Data
Mar. 24, 2019 (IN) 201821048976

(51) Int. Cl. B01L 3/00; G01N 33/70; C12Q 1/32

(52) U.S. Cl. CPC B01L 3/502; G01N 33/70; C12Q 1/32; B01L 2200/025; B01L 2400/0487; B01L 2200/10; B01L 2200/16; B01L 2300/0663; B01L 2300/14; B01L 2200/026

(57) ABSTRACT

A biosensor for detecting analytes present in fluid includes one or more plates configured on a substrate to form at least one channel such that one or more containment chambers are formed in the channels. The channel are mechanically, separated from each other by spacers, and the containment chambers are fluidically separated from adjacent chamber by a discontinuity such that the fluid flows between adjacent chambers only after an application of a predefined pressure on the plate. The multiple chambers allows the fluid to undergo pre-processing using different set of reagents provided at different chambers, to mitigate effects of interferents and to efficiently distribute load of the reagents on the chambers. Further, some of containment chambers allows detection of analytes in the fluid using detection reagents.

### TECHNICAL FIELD

The present disclosure relates to the field of sensors for detection of analytes in a fluid at point of care. More particularly, the present disclosure relates to a disposable biosensor for pre-processing and detection of analytes in a fluid in multiple steps at various spatial locations on the sensor. Further, the present disclosure relates to a biosensor having improved interference removal and reduced reagent load on the spatial locations.

### BACKGROUND

Background description includes information that may be useful in understanding the present invention. It is not an admission that any of the information provided herein is prior art or relevant to the presently claimed invention, or that any publication specifically or implicitly referenced is prior art.

Biosensors are devices used to detect the presence or concentration of analytes such as biomolecules, a biological structure or a microorganism. Biosensor converts these analytes into detectable compounds by action of detection reagents, where these detectable compounds are further detected using detection devices using optical detection, amperometric detection, potentiometric electrostatic piezoelectric electromagnetic, mass sensor, thermo sensors, and the likes.

For instance, in point of care blood testing for detecting analytes present in the blood, interference due to non-specific metabolites is often a major challenge that hampers accuracy of existing analyte detection system. Creatinine being an example of a marker that has very low concentration in blood (40 micromolar (uM)-150 uM, and up to 1000 uM in severe cases), and its detection is subjected to lot of interferences. Further, Serum Creatinine concentration is a primary marker for kidney function testing, and an accurate point of care creatinine measurement is highly desirable for kidney patients.

There are various existing devices for detection of creatinine in whole blood sample. However, they are subjected to various limitations. For instance, the concentration of creatinine is very less in blood (as low as 40 um). This leads to a great susceptibility of some of the existing devices to the interferents whose concentration is very large in blood as compared to creatinine.

The United States Patent Document U.S. Pat. No. 6,767,441B1 discloses an analytical measurement by mediator assisted amperometry. However, the prior art is subjected to interferences due to reaction of mediator/hydrogen peroxide with blood reducing agents like ascorbic acid, bilirubin etc.

The United States Patent Document U.S. Pat. No. 5,200,051A also measures concentration of creatinine in whole blood by direct oxidation of hydrogen peroxide over amperometric electrode. This prior art uses molecular sieves to remove interferents like ascorbic acid etc. However, a large volume of whole blood is required for the device operation and the manufacturing method is relatively complex.

The United States Patent Document US20060228767A1 discloses deployment of reflectance measurement-based system, which removes the interferents by incorporating interference killing enzymes like ascorbic acid oxidase in an auxiliary reagent layer. However, this prior art again requires very large amount of blood (20 ul) and is prone to general inaccuracies of reflectance-based systems. Moreover, the measurement time is very large which is undesirable from point of view of POC testing.

Further, the United States Patent Document US20170284954A1 also mentions detection of creatinine using a set of rare enzymes. However, the prior art does not disclose or suggest how the interferents affect the test results and how their effect can be reduced.

One of the major challenges in detection of creatinine is due to its low concentration in blood, which leads to smaller signals and large impact of variable concentration of interfering metabolites like ascorbic acid, bilirubin, uric acid, hemoglobin, lactic acid etc. Moreover, there is no direct enzyme for conversion of creatinine into an amperometrically detectable compound.

The general set of enzymes used for conversion of creatinine to amperometrically detectable product along with the reaction sets involved are mentioned below: where, the left-hand side of the below reaction sets represents reactant of sequence reaction, the bracketed compound is enabling enzymes and other reagent, the right-hand side representing the product in that enzyme step, and the boldly marked entity represents the amperometrically detected compounds).

Reaction Set 1:

| | |
|---|---|
| Creatinine- (Creatininase)−> Creatinine | 1a |
| Creatinine- (Creatinase)−> Sacrosine | 1b |
| Sacrosine- (Sacrosine Oxidase)−> Hydrogen Peroxide | 1c |

Reaction Set 2:

| | |
|---|---|
| Creatinine- (Creatininase)−> Creatine | 2a |
| Creatine- (Creatinase)−> Sacrosine | 2b |
| Sacrosine- (Sacrosine Oxidase and | 2c |
| Oxidised Mediator)−> Reduced Mediator | |

Reaction Set 1:

| | |
|---|---|
| Creatinine- (Creatininase)−> Creatinine | 3a |
| Creatinine- (Creatinase)−> Sacrosine | 3b |
| Sarcosine- (Sacrosine Oxidase−> Hydrogen Peroxide | 3c |
| Hydrogen Peroxidase-(Peroxidase, Reduced | 3d |
| Mediator)−> Oxidised Mediator + Water | |

The reaction set 1 detects hydrogen peroxide directly, which requires very high voltage capable of oxidizing reducing agents like ascorbic acid and uric acid present in blood. Further, the use of mediator as per the reaction set 2 and 3 allow the use of lower potential and possibility of biamperometry, eliminating the use of reference electrode, thereby, further simplifying the design and fabrication of bio sensor and further reduces the detection potential.

However, in the case of mediator based amperometry there is a further possibility of interference due to degradation of oxidized form of the mediator by reducing agents present in blood. For example, ascorbic acid is known to rapidly react with mediators like ferricyanide, methylene blue and ferrocene to produce reduced form of mediator. Bilirubin and Uric acid are examples of other reducing agents present in blood. The concentration of these analytes in blood can be comparable to or even larger than that of creatinine in whole blood. Thus, they may act as strong interferents and lead to the complete destruction of test result.

Further, in the reaction set 2, there is a further possibility of interference due to dissolved oxygen, in fact the concentration of dissolved oxygen in blood is much larger than concentration of creatinine in normal human blood, and sarcosine oxidase unless modified otherwise has a much stronger affinity towards oxygen as compared to ferricyanide and most of the other mediators. Thus, it is impossible to detect creatinine with this method if dissolved oxygen is not removed.

Furthermore, in the reaction set 3, there is a possibility of interferents like ascorbic acid, hemoglobin, bilirubin etc. to act as a co-substrate to peroxidase and degrade the hydrogen peroxide, thus alternately interfering with the system.

Besides, one thing to note about the existing devices for detecting creatinine in whole blood is that the reaction involves multiple enzymes to be deposited. In order to complete the reaction in shortest possible time, it is required that larger amount of enzymes are deposited on strip. However, due to presence of a large number of enzymes, it is not possible to deposit large quantities of each enzyme without overloading the formulation. Further, the overloaded formulation does not dissolve quickly and uniformly, and thus shows higher variability during amperomtric detection. It should be noted that reagent density of organic film over electrodes also directly influences the response of sensor.

Therefore, there is a need in the art for processing a sample (fluid) in multiple steps at various spatial locations (containment chambers) on a single sensor for quick and enhanced detection of analytes in the sample, mitigating the effects of interferents present in the fluid, and which also have reduced load of reagents on the spatial location.

Various examples of analytes in the background have been illustrated as creatine and creatinine. However, the analytes are not just limited to creatine and creatinine, and all other analytes are well within the scope of the invention.

### Objects of the Present Disclosure

Some of the objects of the present disclosure, which at least one embodiment herein satisfies areas listed herein below.

It is an object of the present disclosure to provide a biosensor and a method for detecting one or more analytes present in a fluid.

It is an object of the present disclosure to process and detect analytes present in a fluid in multiple steps on a single biosensor.

It is an object of the present disclosure to mitigate the effects of interferents and reducing the load of reagents on the channels of a biosensor.

It is an object of the present disclosure to immobilize non-compatible reagents on the biosensor.

It is an object of the present disclosure to detect multiple analytes with same amount of fluid.

It is an object of the present disclosure to provide a biosensor allowing simpler calibration.

It is an object of the present disclosure to provide a biosensor having improved reagent load distribution.

It is an object of the present disclosure to detect analytes present in whole blood where the detection of analytes is subjected to a lot of non-specific interferences.

It is an object of the present disclosure to provide a method to detect creatine, creatinine, and other analytes present in whole blood, mitigating the effects of the interferents.

It is an object of the present disclosure to remove interferents from sample fluid prior to detection of the analytes of the bio sensor.

It is an object of the present disclosure to reduce load of reagents on the channels (containment chambers) of the biosensor, for faster and enhanced processing and detection of the analytes.

### SUMMARY

The present disclosure relates to the field of sensors for detection of analytes in a fluid at point of care. More particularly, the present disclosure relates to a disposable biosensor for pre-processing and detection of analytes in a fluid in multiple steps at various spatial locations on the biosensor. Further, the present disclosure relates to a biosensor having improved interference removal and reduced reagent load on the spatial locations.

It is to be appreciated by a person skilled in the art that the teachings of the present invention is applicable for various embodiments of the present invention where there are more than three containment chamber and channels, and all such embodiments are well within the scope of the present invention.

An aspect of the present disclosure pertains to a biosensor for processing and detecting analytes present in a fluid in multiple steps at various spatial locations (containment chamber) on a single biosensor, where the interferents may be removed in one of the chambers of the biosensor, prior to detection of the analytes in another set of chambers, thereby mitigating the effects of the interferents on the analyte detection process.

In an aspect, the biosensor may comprise a substrate for positioning the various components of the biosensor. The biosensor may comprise a first plate configured on the substrate to form at least one channel between the first plate and the substrate, where the channels are configured to receive the fluid from a first end of the channels and get filled with the fluid. Further, each of the channels may comprise a first containment chamber to facilitate pre-processing of the fluid. In addition, the biosensor may comprise a second plate positioned at a first predetermined distance from the first plate, and configured on the substrate to form a second containment chamber in the channels such that the first containment chamber is fluidically separated from the containment chamber by a first predetermined distance. Furthermore, the fluid may be configured to flow from the first containment chamber into the second containment chamber only when a first predefined pressure is applied on the plates.

In an aspect, the substrate may comprise a set of electrodes that are electrically configured with the detection channel, and may be adapted to be operatively coupled to an amperometric device for detecting and measuring the concentration of the one or more analytes based on a voltage generated across the set of electrodes because of conversion of the one or more analytes into the one or more detectable compounds.

In an aspect, the first containment chamber and the second containment chamber associated with each of the at least one channel may facilitate any or a combination of processing of the fluid, and conversion of one or more analytes present in the fluid into one or more detectable compounds.

In an aspect, the first containment chamber and the second containment chamber may comprise any or a combination of a first set of reagents and a second set of reagents respectively to facilitate any or a combination of processing of the fluid, and conversion of one or more analytes present in the fluid into one or more detectable compounds. The first set of reagents and the second set of reagents may comprise one or more interference removing reagents adapted to remove one or more interferents from the fluid.

Another aspect of the present disclosure pertains to a biosensor for processing and detecting creatinine and creatine present in a biological fluid in multiple steps at various spatial locations (containment chamber) on a single biosensor. At least a set of the containment chambers may be provided with an initial set of reagents (enzymes) comprising creatinase that is slow in action and is required in larger amount, along with the interference removing agents. Further, the other set of containment chambers may be provided only with the detection reagents comprising mediator, sarcosine oxidase, and peroxidise, which are fast in action and are required in small quantity, thereby reducing the load of the reagents on the detection channel and biosensor of the proposed biosensor.

### BRIEF DESCRIPTION OF THE DRAWINGS

The accompanying drawings are included to provide a further understanding of the present disclosure, and are incorporated in and constitute a part of this specification. The drawings illustrate exemplary embodiments of the present disclosure and, together with the description, serve to explain the principles of the present disclosure.

The diagrams are for illustration only, which thus is not a limitation of the present disclosure, and wherein:

FIG. 1 illustrates top view of the proposed sensor for processing and detection of analytes in a fluid in multiple steps at various compartments, in accordance with an embodiment of the present disclosure.

FIG. 2A illustrates top view of a first embodiment of the proposed biosensor for detection of analytes in a fluid, in accordance with an embodiment of the present disclosure.

FIG. 2B illustrates exploded perspective view of the first embodiment of the proposed biosensor with pressers, in accordance with an embodiment of the present disclosure

FIG. 2C illustrates top view of a second embodiment of the proposed biosensor with controlled processing and amperometric transduction, in accordance with an embodiment of the present disclosure

FIG. 2D illustrates exploded perspective view of the second embodiment of the proposed biosensor with pressers, in accordance with an embodiment of the present disclosure.

FIG. 3 illustrates exploded perspective view of a third embodiment of the proposed biosensor having containment chambers of different thickness, in accordance with an embodiment of the present disclosure.

FIG. 4A illustrates top view of a fourth embodiment of the proposed biosensor having independent processing in each channel, in accordance with an embodiment of the present disclosure.

FIG. 4B illustrates exploded view of the fourth embodiment of the proposed biosensor with pressers, in accordance with an embodiment of the present disclosure.

FIG. 5A illustrates top view of a fifth embodiment of the proposed biosensor with multiple containment chambers, in accordance with an embodiment of the present disclosure

FIG. 5B illustrates exploded view of the fifth embodiment of the proposed biosensor with pressers, in accordance with an embodiment of the present disclosure.

### DETAILED DESCRIPTION

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

Various terms as used herein are shown below. To the extent a term used in a claim is not defined below, it should be given the broadest definition persons in the pertinent art have given that term as reflected in printed publications and issued patents at the time of filing.

In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all groups used in the appended claims.

The present disclosure relates to the field of sensors for detection of analytes in a fluid at point of care. More particularly, the present disclosure relates to a disposable biosensor for pre-processing and detection of analytes in a fluid in multiple steps at various spatial locations on the biosensor. Further, the present disclosure relates to a biosensor having improved interference removal and reduced reagent load on the spatial locations.

According to an aspect, the present disclosure elaborates upon a sensor for detection of analyte in a fluid, the sensor comprising: a substrate; a first plate configured on the substrate to form at least one channel between the first plate and the substrate, each of the at least one channel can include a first containment chamber configured to receive and get filled with the fluid, a second plate positioned at a first predetermined distance from the first plate, and which can be configured on the substrate to form a second containment chamber in each of the at least one channel, wherein the containment chamber and the second containment chamber of each of the at least one channel can be fluidically separated from each other such that the fluid is configured to flow between the corresponding containment chambers only when a predefined pressure is applied on the corresponding plate; and wherein the each of the at least one channel can be mechanically separated from each other; and wherein the first containment chamber and the second containment chamber associated with each of the at least one channel can facilitate conversion of one or more analytes present in the fluid into one or more detectable compounds; and wherein the sensor can be configured to be operatively coupled to a detection device selected from a group comprising optical device, electrochemical device, electrostatic device, piezoelectric device, electromagnetic device, and any or a combination of a thermal sensor, and a mass sensor.

In an embodiment, the sensor can include at least one spacer configured between the substrate, and the first plate and the second plate to form the first containment chamber and the second containment chamber respectively.

In an embodiment, the at least one spacer can include: a first set of spacers having a first predefined thickness, and positioned between the first plate and the substrate to form the corresponding first containment chamber of the at least one channel, having the first predefined thickness; and a second set of spacers having a second predefined thickness, and positioned between the second plate and the substrate to form the corresponding second containment chamber of the at least one channel, having the second predefined thickness.

In an embodiment, the sensor can be adapted to be configured with at least one presser, and wherein the at least one presser can be configured to facilitate application of pressure on at least one plate to enable flow of the fluid between the corresponding containment chambers.

In an embodiment, the sensor can include at least one third plate positioned at a second predefined distance from the second plate, and configured over the substrate to form a third containment chamber in each of the at least one channel.

In an embodiment, the third containment chamber can be fluidically separated from the corresponding second containment chamber such that that the fluid can be configured to flow between the third containment chamber and the second containment chamber only when the predefined pressure is applied on at least one of the corresponding plates.

In an embodiment, the first containment chamber and the second containment chamber can include any or a combination of a first set of reagents and a second set of reagents respectively to facilitate any or a combination of processing of the fluid, and conversion of one or more analytes present in the fluid into one or more detectable compounds, and wherein the first set of reagents can include one or more interference removing reagents adapted to remove one or more interferents from the fluid.

In an embodiment, the one or more interferents can include Absorbic acid, and wherein the one or more interference removing reagents comprises Ascorbate Oxidase, Ascorbic Acid Oxidase, Glucose Oxidase, and oxidized from of the mediators.

In an embodiment, the one or more analytes can include any or a combination of glucose, cholesterol, HbA1C, protein, and bilirubin, and wherein the one or more interferent comprises any or a combination of bilirubin, oxygen, glucosem, proteins, urea, and uric acid, and wherein the one or more interference removing reagents comprises any or a combination of oxidizing agents, reducing agents.

In an embodiment, one of the containment chamber can include a first agent that reacts with at least one component present in the fluid or produces a second agent that reacts with at least one component present in the fluid, and wherein another containment chamber comprises a third agent that removes any or a combination of the first agent and the second agent from the fluid.

In an embodiment, the first agent, the second agent and the third agent can be selected from a group comprising ferricyanide, ferrocyanide, hydrogen peroxide, oxygen, an oxidising agent, a reducing agent, an electrochemical mediator, an enzyme, glucose, urea, and uric acid.

In an embodiment, the first agent can be an electro-chemical mediator and the third agent can be an enzyme

In an embodiment, the first agent can be ferricyanide, and the third agent can be glucose dehydrogenase.

In an embodiment, one of the containment chambers corresponding to a first channel among the at least one channel can include a predetermined quantity of the analyte or its derivative, wherein measurement for the analyte in the fluid and measurement for the analyte in the fluid enriched with derivative is made.

In an embodiment, a consecutive containment chambers in a second channel among the at least one channel allows detection of a different analyte. Alternatively, multiple consecutive containment chamber in the same channel can be used for detection of multiple analytes present in fluid, using detection methods including any or a combination of optical, electrochemical, mass based, thermal detection to help in reduction of total volume of sample, which may be important in many cases. In a single channel with same volume of fluid repetitively being used in different chambers for detection of different analytes. This is to decrease total volume of fluid required for detection of an analyte.

According to another aspect, the present disclosure elaborates upon a method for detection of an analyte in a biological fluid, the method including: retaining, at a first spatial region, the biological fluid for a predefined period of time, wherein the first spatial location allows a first set of reactions to be performed on the biological fluid; and transferring, the biological fluid from the first spatial region to a second spatial region, wherein movement of the fluid from the first spatial region to the second spatial region is controlled, converting, at a second spatial location, the analytes present in the biological fluid into a final detectable entity.

In an embodiment, the the first set of reaction can include creatinine conversion to creatine and creatine conversion to sarcosine.

According to another aspect, the present disclosure elaborates upon a sensor for detection of at least one analyte in a fluid, the sensor can include at least one channel, wherein at least one of the channels can include at least one stopper in order to contain the fluid such that at least one containment chamber is formed in each of the at least channel; and wherein, the at least one of the containment chambers can include any or a combination of fluid processing, analyte detection, sample pretreatment, interference removal, analyte enrichment, dissolved oxygen removal, dissolved oxygen enrichment, analyte derivative enrichment, partial reaction completion; and wherein the at least one stopper can include any or a combination of capillary break, a hydrophobic coating, a discontinuity in hydrophillic coating, and wherein transfer of sample between consecutive containment chamber separated by stopper is controlled externally by a device provided with the bio sensor.

In an embodiment, the first agent second agent and the third agent can be selected from a group comprising ferricyanide, ferrocyanide, hydrogen peroxide, oxygen, an oxidising agent, a reducing agent, an electrochemical mediator, an enzyme, glucose, urea, and uric acid.

In an embodiment, the first agent can be ferricyanide, and the third agent can be glucose dehydrogenase.

In an embodiment, the one of the containment chambers corresponding to a first channel among the at least one channel can include a predetermined quantity of the analyte or its derivative, wherein measurement for the analyte in the fluid and measurement for the analyte in the fluid enriched with analytes or its derivative is made.

In an embodiment, a consecutive containment chambers in a second channel among the at least one channel can allow detection of a different analyte. Alternatively, multiple consecutive containment chamber in the same channel can be used for detection of multiple analytes present in fluid, using detection methods including any or a combination of optical, electrochemical, mass based, thermal detection to help in reduction of total volume of sample, which may be important in many cases. In a single channel with same volume of fluid repetitively being used in different chambers for detection of different analytes. This is to decrease total volume of fluid required for detection of an analyte.

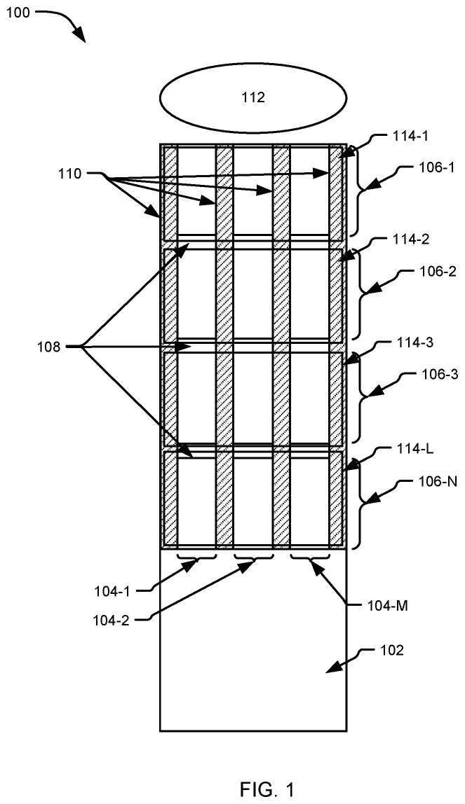

FIG. 1 illustrates top view of the proposed sensor for processing and detection of analytes in a fluid in multiple steps at various compartments, in accordance with an embodiment of the present disclosure.

As illustrated in FIG. 1, the proposed sensor 100 for detection of analyte in a fluid can include a substrate 102. Further, the sensor 100 can include a first plate 114-1 which can be configured on the substrate 102 to form at least one channel (104-1 to 104-N) (collectively referred to as channels 104, herein) between the first plate 114-1 and the substrate 102. Each of the at least one channel 104 can include a corresponding containment chamber such that a first containment chamber 106-1 is formed below the first plate, which can be configured to receive and get filled with the fluid 112. In an embodiment, a second plate 114-2 can be positioned at a first predetermined distance from the first plate 114-1, and which can be configured on the substrate 102 to form a second containment chamber (106-2) below the second plate 114-2 in each of the at least one channel (104). In another embodiment, the sensor 100 can include a set of third plates 114-3 to 114-L positioned at a second predefined distance from the second plate 114-2, and configured over the substrate to form multiple containment chambers in each of the at least one channel 104, such that containment chambers 106-3 to 106-N are formed in the channels 104, below the each of the third plates 114-3 to 114-L.

In an embodiment, the first containment chamber 106-1 and the second containment chamber 106-2 of each of the at least one channel 104 can be fluidically separated from each other through discontinuities 108 such that the fluid is configured to flow between the corresponding containment chambers 106-1 and 106-2 only when a predefined pressure is applied on the corresponding plate. In another embodiment, each of the at least one channel 104 can be mechanically separated from each other by at least one spacer 110.

In an embodiment, the third containment chamber 106-3 can be fluidically separated from the corresponding second containment chamber 106-2 such that that the fluid can be configured to flow between the third containment chamber 106-3 and the second containment chamber 106-2 only when the predefined pressure is applied on at least one of the corresponding plates 114.

In an embodiment, the sensor 100 can include at least one spacer 110 configured between the substrate 102, and the plates to form the containment chambers 106 and the channels 104.

In an embodiment, the sensor 100 can be adapted to be configured with at least one presser, which can be configured to facilitate application of pressure on at least one of the plates 114 to enable flow of the fluid between the corresponding containment chambers 106.

In an exemplary embodiment, the spacer 110 can be formed by PET or polyester-backed acrylic adhesive double-sided adhesive tapes preferably 60-150 microns, made from a dielectric material selected from PET, acrylic, polycarbonate, but not limited to the likes. The spacer can also act as barrier between multiple channels

In another exemplary embodiment, the spacer 110 can be a non-adhesive sheet of the predetermined thickness, which can be placed between the substrate 102 and the plates 114 to form an assembly. Further, the assembly can be fitted compactly in a casing to form the biosensor 100.

In an embodiment, the containment chambers 106 can include any or a combination of a first set of reagents and a second set of reagents to facilitate any or a combination of processing of the fluid 112, and conversion of one or more analytes present in the fluid 112 into one or more detectable compounds (detectable entity). In an embodiment, the first set of reagents can include one or more interference removing reagents adapted to remove one or more interferents from the fluid.

In an embodiment, the fluidic separation 108 between the consecutive containment chambers can be provided by a means including any or a combination of capillary break and hydrophobic barrier.

In an embodiment, the sensor 100 can be configured to be operatively coupled to a detection device which can be selected from a group including optical device, electrochemical device, electrostatic device, piezoelectric device, electromagnetic device, and any or a combination of a thermal sensor, and a mass sensor, but not limited to the likes.

In an implementation, the proposed sensor 100 can provide a separate containment chamber for modification or preprocessing of the fluid prior to detection of the analytes in another separate containment chambers. The fluid can be preprocessed/incubated for a desired amount of time in the first containment chamber 106-1, and can be later transferred to the second containment chamber 106-2 or any other containment chamber 106-3 to 106-N for further reactions and/or subsequent detection of the analytes in the fluid.

In another implementation, when the detection is through optical devices, where chemical formulation (reagents) is required to be deposited over a membrane and when the fluid drains over it, the analyte is converted into final optically active component (usually a dye or other colored compound). The optical devices can further measure a corresponding reflectance, which is proportional to concentration of the analytes. However, many a times, the formulation (reagents) to be deposited might not be suitable for the membrane. For instance, a formulation of reagents having very high pH might degrade the membrane over time. In another instance, a formulation of reagent can be very viscous, and can have very high concentration of certain components that might not get uniformly spread onto detection membrane. In order to solve the above problem, the formulation reagents which is not compatible with membranes can be easily deposited in the containment chambers of the present bio sensor. And once the required steps are completed, the fluid can be transferred to membrane which is affixed in one of the chambers as detection element. In case membrane is affixed as a detection element, it might not be necessary to have a corresponding top plate 114.

In another implementation, when detection method being employed is amperometric, the analyte is converted into an electrochemically active compound which might undergo redox conversion upon application of a potential, to generate a corresponding current that is proportional to the analyte concentration. However, it might be required that no formulation reagent is deposited over the electrodes of the biosensor, as if the formulation reagent is deposited over the electrodes, generally a layer of deposition might not get completely dissolve in the fluid upon introduction of the fluid, and they get sustain over electrodes during measurement, changing the diffusion coefficients of final redox species near electrode surface and hence affecting the current measurement or analyte concentration measurement process.

In order to solve this problem, the present invention provides a separate channel (or containment chamber) preceding the containment chamber having the electrodes, where the detection formulation can be deposited, and the final product is formed such that electrodes are formulation reagents free or have a minimal chemical deposition on it.

In another implementation, in order to convert the analyte into final detectable entity, a sequence of chemicals reactions is required, each with its own set of catalysts, reagents and optimal pH condition. It is often possible that conditions and chemicals required by a particular step in the sequence is not compatible with those of others. For example, a first step in the sequence requires pH to be acidic but second step might contain an enzyme that is deactivated in acidic medium. It again desirable in such situations that two incompatible reactions take place in different channels. The present biosensor 100 provides a separate set of containment chambers for a first set of reactions to be performed, the other set of containment chambers for a second set of reactions to be performed, as a result, the two incompatible sets of reactions can be performed in two separate set of containment chambers.

In another implementation, the interferents can be removed in the any of containment chambers by performing electro-oxidation and/or reduction in the corresponding containment chambers.

In another implementation, one of the containment chambers can include dissolved oxygen generating reagents which can generate oxygen after coming in contact with fluid to enrich the fluid with dissolved Oxygen. The containment chamber can facilitate electrochemical oxidation of the fluid to convert water into Oxygen.

The present disclosure also elaborates upon a method for detection of creatinine present in a fluid. The method can include a first step of converting, at a first spatial location, creatinine present in the biological fluid into sarcosine. The method can further include a second step of controlled transferring of the sarcosine from the first spatial location to second spatial location. The method can further include a third step of converting, at a second spatial location, the sarcosine into a final detectable entity.

In an implementation, the proposed method can be implemented using the present bio sensor, where the creatine present in the fluid can be converted into sarcosine in the first containment chamber 106-1. Further, the sarcosine can be transferred from the first containment chamber to the second containment chamber 106-2 in a controlled manner by application of a predefined force on the first plate over the first containment chamber 106-1. Furthermore, the sarcosine can be converted into final detectable entity in the second containment chamber 106-2 by action of a set of detection reagents.

It is to be appreciated that the proposed method for detection of creatine or creatinine in the biological fluid can also be implemented using any bio sensor regardless of their structure, and all such aspect of the proposed method are well within scope of the present disclosure.

In an implementation, the biosensor 100 can be calibrated by providing a known concentration of analyte or its derivative in one of the containment chambers of one of the channels which can be different than primary channel. The analytes can undergo preprocessing in one of the containment chambers followed by detection in the other containment chambers, and finally the concentration of the analytes can be measured using any of the known methods and devices. The difference between the signals sensed by the detection devices in the original fluid, a blank, and the sample with increased analytes can be processed to provide a calibration curve for signal vs concentration. A Calibration->y(current)=mx(concentration)+c. c in the matrix is determined by measuring blank. The m is determined by deposition of known amount of analyte or its derivative in a containment chamber prior to detection in a separate channel. This way we have two data points to determine m and c, thereby facilitating calibration of the biosensor. Such on-chip calibration method can be desirable for fluids with complex matrix like whole blood containing erythrocytes, many metabolites and proteins and other biological fluids like saliva, urine etc

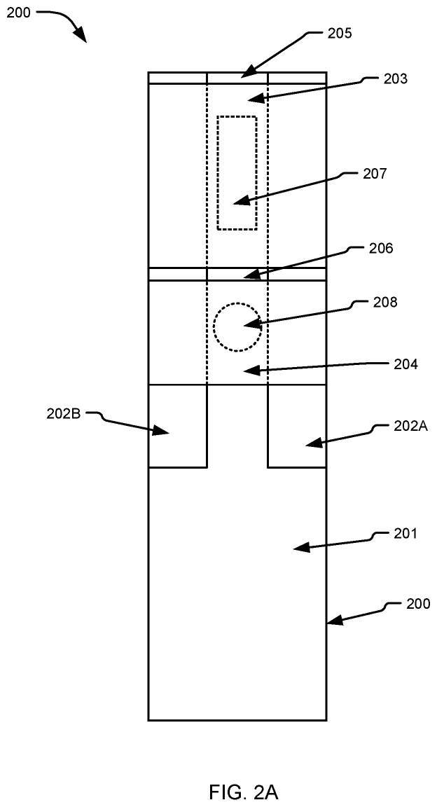

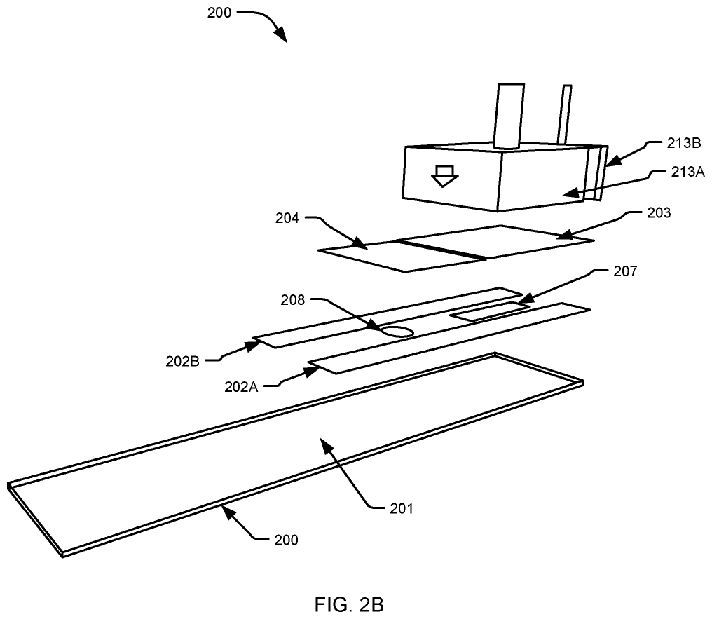

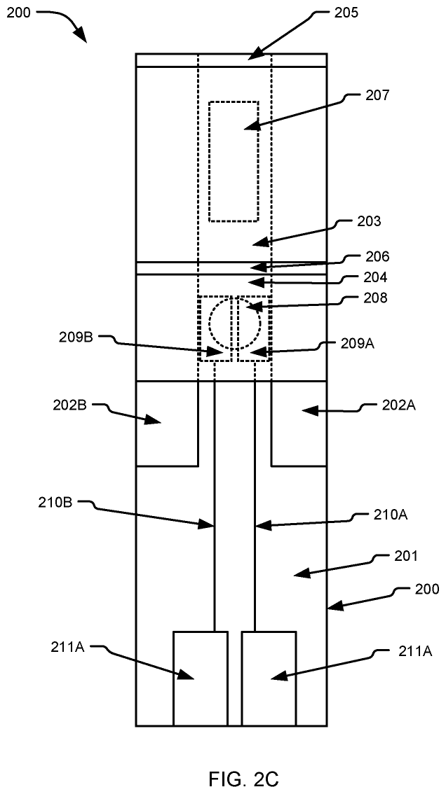

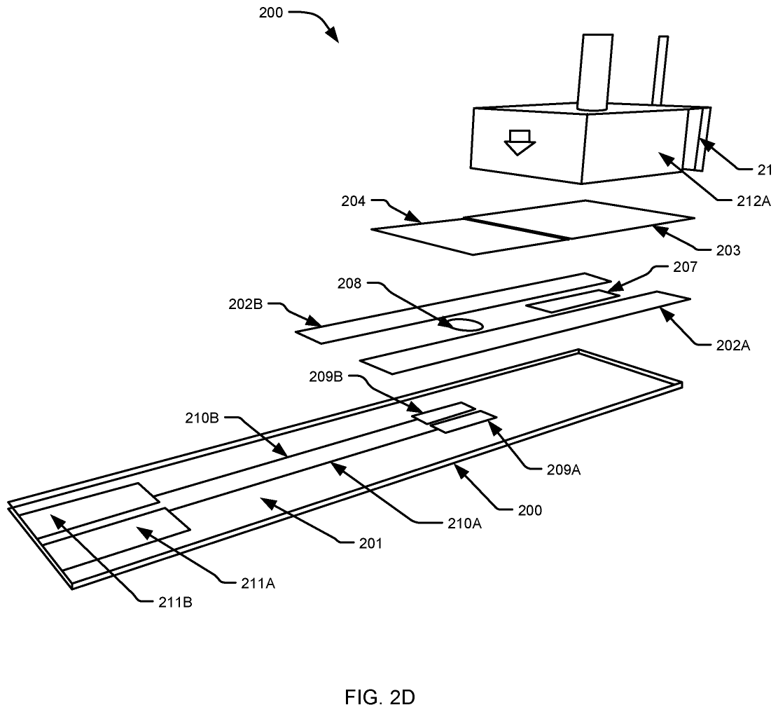

FIG. 2A illustrates top view of a first embodiment of the proposed biosensor for detection of analytes in a fluid, in accordance with an embodiment of the present disclosure.

FIG. 2B illustrates exploded perspective view of the first embodiment of the proposed biosensor with pressers, in accordance with an embodiment of the present disclosure

As illustrated in FIGS. 2A and 2B, in an aspect, the proposed biosensor 200 for detecting analytes in a fluid, can include a first plate 203 configured on the substrate 201 to form at least one first channel (also referred to as channel, herein) between the first plate 203 and the substrate 201. The channel can be configured to receive the fluid from an inlet port 205 configured at a first end of the channels. The channel can get filled with the fluid due to capillary action. The fluid can include any or a combination of blood, plasma, and serum, but not limited to the likes. The fluid can be inserted in the channel through the inlet port 205, which allows the fluid to fill the channels due to capillary action. The channels can include a first set of reagents 207 (also referred to as first reagents, or first reagents formulation 207, herein) to facilitate processing of the fill fluid. The first reagents 207 can include one or more interference removing reagents (also referred to as interference removing reagents, herein) adapted to remove one or more interferents (also referred to as interferents, herein) from the fluid.

In an embodiment, the biosensor 200 can include a second plate 204 positioned at a first predetermined distance from the first plate 203, and configured on the substrate 201 to form a second compartment in the at least one channel such that the first containment chamber is fluidically separated from the second containment chamber by a first predetermined distance. The fluid filling in the channel stops at a discontinuity 206 due to the fluidic separation between the first containment chamber and the second containment chamber. The fluid can be configured to flow from the first containment chamber into the second containment chamber only when a first predefined pressure is applied on the corresponding plates. This enables the fluid to be retained in the first containment chamber for a predefined time to allow the preprocessing of the fluid as well as allowing the interference removing agents to remove the interferents from the fluid in the first compartment itself, prior to being transferred to the second containment chamber for detection of the analytes.

In an embodiment, the second containment chamber can include one or more detection reagents 208 (also referred to as detection reagents or detection reagents deposition 208, herein) that are adapted to convert analytes present in the fluid into one or more detectable compounds (also referred to as detectable entity, herein).

In an embodiment second containment chamber can include detection elements like membranes, electrodes, peizoelectric sensor, thermal sensors, etc depending on nature of detection.

In an embodiment, presser 213A, 213B can be adapted to be configured with the first plate 203 of the biosensor 200. The presser can be configured to facilitate application of pressure on the first plate 203 such that the fluid flows from a second end of the first containment chamber into the second containment chamber, however, the flow of the fluid towards the first end or inlet port 205 of the first compartment can be controlled.

In an embodiment, in order to make sure that the fluid does not flow out of the inlet port 205, when the presser 213A compresses the first plate 203, before it does so, the presser 213B which is much smaller in area as compared to the presser 213A can be pressed against the plate at a point that is closer to the inlet port. The presser 213B can block the backflow of the fluid to the inlet port when the presser 213A compresses the plate. Thus, the only direction in which fluid can be displaced is towards the second containment chamber. Another advantage of having the presser 213B is that the fluid once transferred to the second containment chamber does not recede into the first containment chamber. Further, when the presser 213B presses the first containment chamber, it would displace a small amount of the fluid, however, this volume (small amount) can be minimized by minimizing area of the presser 213B as compared to that of the first containment chamber.

In an embodiment, the first reagents 207 and the detection reagents 208 can be provided in the first containment chamber and the second detection chamber respectively, in a dry state, but not limited to the like.

In an embodiment, the substrate 201 can be any flexible or rigid dielectric material, which can be selected from a material including polyamide, epoxy, and PET, but not limited to the likes. In another embodiment, the substrate 201 can be selected from a transparent and opaque material based on the requirement of biosensor and type of detection device involved. For instance, the material can be transparent and/or opaque when amperometric detection devices are being implemented. However, when optical detection technique and devices are involved, then the material can be transparent.

In an embodiment, the biosensor can include at least one spacer 202A,B (also referred to as spacer, herein) configured between the substrate 201, and the first plate 203 and the second plate 204 to form the channels. In an exemplary embodiment, the spacer 202A,B can be made a double-sided material selected from PET, and polyester, but not limited to the likes. In an exemplary embodiment, the spacers can be double-sided PET/polyester-based adhesive tapes 20 um-200 um thick depending on the biosensor requirement.

In another exemplary embodiment, the presser can be a single structure having slanted base made of a flexible material including rubber and the likes, such that the presser firstly applies a force at a first side of the corresponding plate towards which the flow of the flow is to be prevented, and then upon further application of the force, the presser enables flow of the fluid in a direction opposite to the first side of the plate.

In an exemplary embodiment, as per reaction set 3, the first reagents (also referred to as first deposition 207) in the first containment chamber can include an initial set of enzymes including creatininase and creatinase, and the detection reagents (also referred to as detection channel deposition 208) in the second containment chamber can include sarcosine oxidase, peroxidase and reduced form of an electrochemical mediator. This is preferable mode of distribution of reagents as creatinase is relatively very slow enzyme and large quantity of it may be required for reaction for conversion of creatinine to sarcosine and thereon. This large quantity of creatinase if deposited over electrodes (if implemented for amperometric detection devices) in the second compartment, may interfere with the accuracy of amperometric response. Moreover, if sarcosine oxidase and onwards of it are not included in the first containment chamber, the impact of interferents on the reaction system is minimized as the major mechanism of the interference is reaction of redox species in sample with peroxide, or reduced or oxidized form of the mediator, and concentration of these interferents is majorly reduced during preprocessing in the first containment chamber.

In another exemplary embodiment, as per reaction set 2, the first deposition 207 in the first containment chamber can include creatinase and creatininase along with a dissolved oxygen killing compound like glucose oxidase and other interference killing compounds like ascorbate oxidase and bilirubin oxidase etc. Further, the detection reagents 208 in the other containment chamber can include sarcosine oxidase and oxidized form of the mediator. It is important here that glucose oxidase does not dissolve in the fluid during pre-processing as it might react with mediators present in the second containment chamber and interfere with the result. In this particular embodiment, there is another advantage that hydrogen peroxide is not produced during the detection sequence, thus the peroxide related interferences are absent.

Experimental Results to Examine the Removal of Absorbic Acid Interference Using the Proposed Biosensor:

In an implementation, the substrate 201 was PCB based ENIG gold coated electrode. Spacer used were polyester based tapes 110 microns thick, that were laser cut into channels of width 3.5 mm and length 13 mm to accommodate two containment chambers of a single channel. The length of first containment chamber was 7 mm and second containment chamber was 4 mm with 1 mm of gap between two. The chambers were formed by adhering a polyester sheet (0.25 mm) of width 7 mm and 4 mm respectively In an implementation, the following parameters were considered during the experiments and current was measured at the electrodes:
1. Spacer thickness: 110 microns.
2. Formulation 207 (5 microlitre (uL) inside the first containment chamber): 2 mg/ml pullulan+1 mg/ml ascorbic acid.
3. Formulation 208 (3 microlitre (uL) inside the second containment chamber): pullulan (2 mg/ml as a film-forming agent)+Sarcosine oxidase (procured from Kikkoman 5 mg/ml), HRP (1 mg/ml)+ferrocyanide (50 mM).
4. Area of electrodes: 6 mm²
5. Time in first containment chamber: 100 seconds

The test results obtained are mentioned below:

| | Time in first | |
|---|---|---|
| | containment | Current measured |
| Solution for testing | chamber | (nano Amperes) |
| Sarcosine 50 uM | 0 seconds | 170 |
| Sarcosine 50 uM | 100 seconds | 175 |
| Sarcosine 50 uM Ascorbic acid 100 uM | 0 seconds | 75 |
| Sarcosine 50 uM Ascorbic acid 100 uM | 100 seconds | 175 |

Clearly the interference due to ascorbic acid is completely removed when the solution containing ascorbic acid is preprocessed and the sample is degraded of ascorbic acid using Ascorbate oxidase as interference removing agent.

It is to be appreciated that the above-demonstrated experiment is implemented for removal of ascorbic acid interference in a two-electrode amperometric system from measurement of sarcosine. However, a similar experiment can be performed for other transduction like 3 electrode amperometric detection, photometric detection for any other analyte not limited to glucose, cholesterol, HbA1C, protein, bilirubin etc for removal of any other interferent like bilirubin, oxygen, glucosem, proteins, urea, uric acid.

Experimental Results to Examine the Improved Detection of Creatine Using the Proposed Biosensor and Method Compared to Conventional Biosensor an Dmethod Having No First Compartment for Pre-Processing:

In an implementation, the following parameters were considered during the experiments and current was measured at the electrodes:
1. Spacer thickness: 110 microns.
2. Formulation 207 (5 microlitre (uL) inside the first containment chamber): pullulan (2 mg/ml as a film-forming agent)+Creatininase (procured from Kikkoman 1 mg/ml) creatinase (procured from Kikkoman 1 mg/ml).
3. Formulation 208 (3 microlitre (uL) inside the second containment chamber): pullulan (2 mg/ml as a film-forming agent)+Sarcosine oxidase (procured from Kikkoman 5 mg/ml), HRP (1 mg/ml)+ferrocyanide (50 mM).
4. Area of electrodes: 6 mm²
5. Time in first containment chamber: 100 seconds

The test results obtained are mentioned below:

| | First containment | Current measured |
|---|---|---|
| Solution for testing | chamber time | (nano Amperes) |
| Sarcosine 50 uM | 0 | 180 |
| Creatinine 50 uM | 100 | 176 |
| Creatinine 100 uM | 100 | 252 |
| Creatinine 200 uM | 100 | 450 |
| Creatinine 300 uM | 100 | 608 |

Further, comparison was done between extents of interference in traditional single containment chamber mode vs proposed multiple containment chamber mode biosensor (present invention). In conventional single containment chamber mode, all reagents were deposited into a single containment chamber. Thus, detection time was 60 seconds and there was no preprocessing. Whereas in proposed multiple containment chamber mode as disclosed in present invention, the above-mentioned formulations 207, 208 were used. The detection zone (second containment chamber) time required was only 5 seconds and preprocessing zone (first containment chamber) time was 100 seconds Description of experimental setup:

### Single Containment Chamber Mode

| | |
|---|---|
| First containment | No deposition |
| chamber | |
| Second containment | Creatinase(20 mg/ml) + creatininase(1 |
| chamber | mg/ml) + sarcosine oxidase (5 mg/ml) + |
| | peroxidase(1 mg/ml) + pullulan (2 mg/ml) |
| Method | Creatinine solution is inserted directly in |
| | detection channel, and allowed to stand for |
| | 60 seconds and then potential of 0.2 V is applied. |

### Multiple Containment Chamber Mode (Present Invention)

| | |
|---|---|
| First containment | Creatinase + creatininase |
| chamber | |
| Second containment | Sarcosine oxidase + peroxidase |
| chamber | |
| Method | Solution is inserted in second containment |
| | chamber, and allowed to stand for 100 seconds |
| | and then transferred to second containment chamber, |
| | where it stands for 5 seconds for second containment |
| | chamber reaction completion and then potential |
| | of 0.2 V is applied. |

### Results Obtained

| | Single containment | | | |
|---|---|---|---|---|
| | chamber Mode- | | Current (nA)- multiple | |
| | represents existing state | | containment chamber | |
| | of art | | mode- proposed novelty | |
| | Measured | % | Measured | % |
| Solution | current(nA) | interference | Current | interference |
| Blank | 41 | — | 52 | — |
| 50 uM creatinine | 186 | 0 | 177 | 0 |
| 50 uM creatinine + 20 uM | 131 | 38 | 148 | 23 |
| Ascorbic Acid | | | | |
| 50 uM creatinine + 50 uM | 48 | 95 | 147 | 23 |
| Ascorbic acid | | | | |
| Time of interaction of | 60 seconds | | 5 Seconds | |
| ferricyanide with ascorbic acid | | | | |

Based on the above experimental results, it is clear that the overall interference due to ascorbic acid is reduced in the multiple containment chamber (present invention) where during the slow reactions were completed in the first containment chamber itself.

FIG. 2C illustrates top view of a second embodiment of the proposed biosensor with controlled processing and amperometric transduction, in accordance with an embodiment of the present disclosure

FIG. 2D illustrates exploded perspective view of the second embodiment of the proposed biosensor with pressers, in accordance with an embodiment of the present disclosure.

As illustrated in FIGS. 2C and 2D, in an embodiment, the second embodiment of the proposed biosensor 200 can include a set of electrodes 211A.B that can be electrically configured with the detection channel. The set of electrodes 211A,B can be positioned on the substrate and can be adapted to be operatively coupled to an amperometric detection device. The conversion of the analytes into the detectable compounds can generate a current, corresponding to concentration of the detected analytes, across the set of electrodes. The amperometric device can be configured to measure the concentration of the analytes based on the current generated between the set of electrodes.

In an exemplary embodiment, the spacer 202A,B can be formed by PET or polyester-backed acrylic adhesive double-sided adhesive tapes 60-150 microns. The first plate 203 and the second plate 204 can be a deformable sheet of 0.1-0.2 mm thickness, made from a dielectric material selected from PET, acrylic, polycarbonate, but not limited to the likes.

In another exemplary embodiment, the spacer 202A,B can be a non-adhesive sheet of the predetermined thickness, which can be placed between the substrate 201 and the plates to form an assembly. Further, the assembly can be fitted compactly in a casing to form the biosensor 200.

The fluid in the first containment chamber can be subjected to modification by the first reagents, which can be deposited on a lower or upper or both the faces of the channels. The first reagents 207 can include the interference removing agents, as well as an initial set of enzymes such that the initial part of the reaction is completed. In an exemplary embodiment, the interference removing agents can be interference reducing enzymes like ascorbic acid oxidase, bilirubin oxidase, but not limited to the likes. In another exemplary embodiment, the interference removing agents can include oxidizing agents which can reduce the total concentration of interferents. In yet another exemplary embodiment the interference removing agent can include the oxidized form of mediator dissolved in a non-dissolving formulation. These interference removing agents can be incorporated as a part of the first reagents along with other necessary components like dissolving polymers, salts, buffer and surfactants, and the likes.

In an implementation, upon sufficient time in the first containment chambers, the fluid can be transferred to the second containment chambers formed between the second plate 204 and the substrate 201.

In an embodiment, the detection reagents 208 can include a final set of enzymes and mediators which can convert the analytes into amperometrically detectable products. This conversion can generate a constant potential (eg. 0.15-0.45 V) across the set of electrodes 209A,B through pads 211A,B connected to electrodes by connecting lines 210A,B. The generated potential can be measured by the electronic detection devices, which is proportional to concentration of amperometrically detectable compounds, which in turn is proportional to concentration of creatinine (analytes) in the fluid.

In an embodiment, the set of electrodes 209A, B can enable electro-chemical oxidation of the one or more interferents to remove them from the fluid I nay of the containment chambers.

In an implementation, Ferricyanide and other oxidizing agent in a non-dissolving film can be provided in the first containment chamber, such that the Ferricyanide and other oxidizing agents can get completely dissolved in first containment chamber and then in next containment chamber any excess Ferricyanide and other oxidizing agent can be completely reduced.

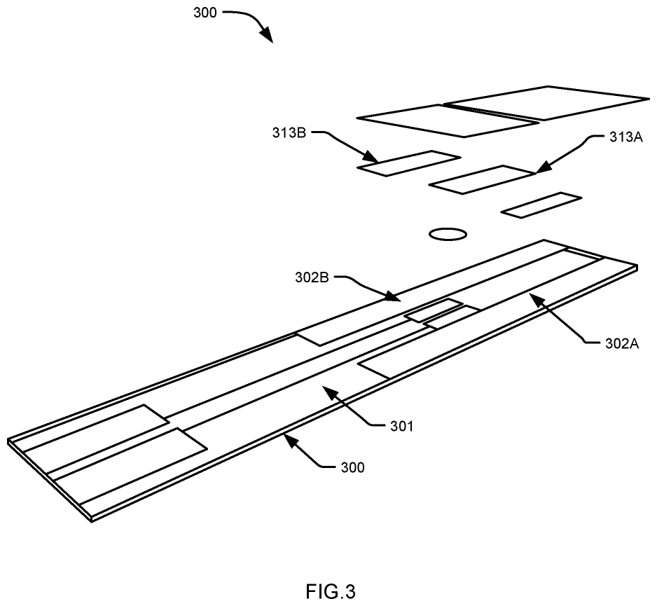

FIG. 3 illustrates exploded perspective view of a third embodiment of the proposed biosensor having containment chambers of different thickness, in accordance with an embodiment of the present disclosure.

As illustrated in FIG. 3, in a third embodiment of the proposed biosensor 300, the at least one spacer can include a first set of spacers 302A,B having a first predefined thickness, and positioned between the first plate and the substrate 301 to form the first compartment in the channels having the first predefined thickness. Further, the at least one spacer can include a second set of spacers 313A,B having a second predefined thickness, and positioned between the second plate and the substrate 301 to form the second containment chamber in the channel having the second predefined thickness.

Before the fluid is transferred to the detection channel, it is desirable that the reaction in the first compartment is completed and the intermediary products are homogeneously distributed. For reaction to complete, the complete amount of creatinine should diffuse to surface of deposition and sarcosine produced on surface should homogeneously distribute to whole sample.

In an implementation, the time required for completion of diffusion is directly related to the square of the distance. Thus, lowering the thickness of the compartments can greatly decrease the completion time for homogenization through diffusion. However, one cannot keep on decreasing this thickness as per previous embodiments of the present invention, The main restriction being put by the minimum value of thickness required to maintain a thickness independent amperometric response. Typically, a spacing >80 microns is required for amperometric biosensors.

In an exemplary embodiment as illustrated in FIG. 3, the first containment chamber can have the single set of spacers 302A,B of thickness ranging from 25-40 microns, and the second containment chamber can include the second set of spacers 313A,B with a thickness of 40-60 microns. This can facilitate the reaction in the first containment chamber to be completed in much smaller time as compared to the second embodiment of the proposed biosensor as illustrated in FIG. 2. One should note that in such a case the length of the channel has to be also increased accordingly.

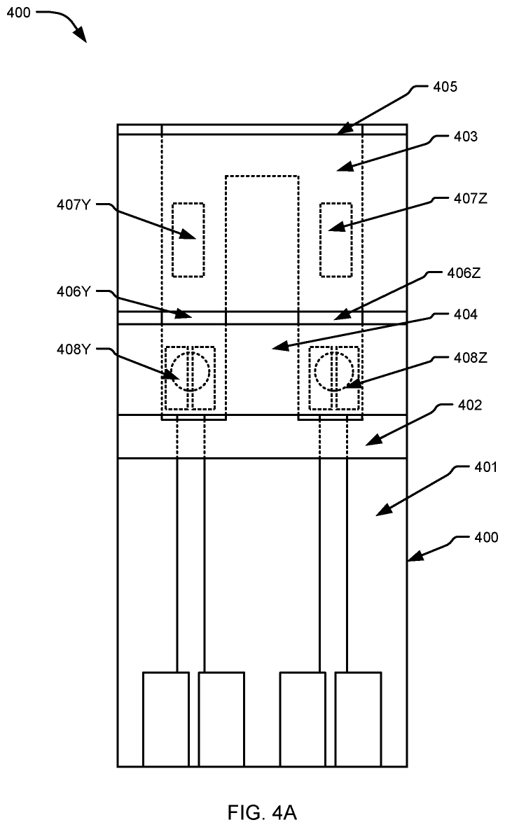

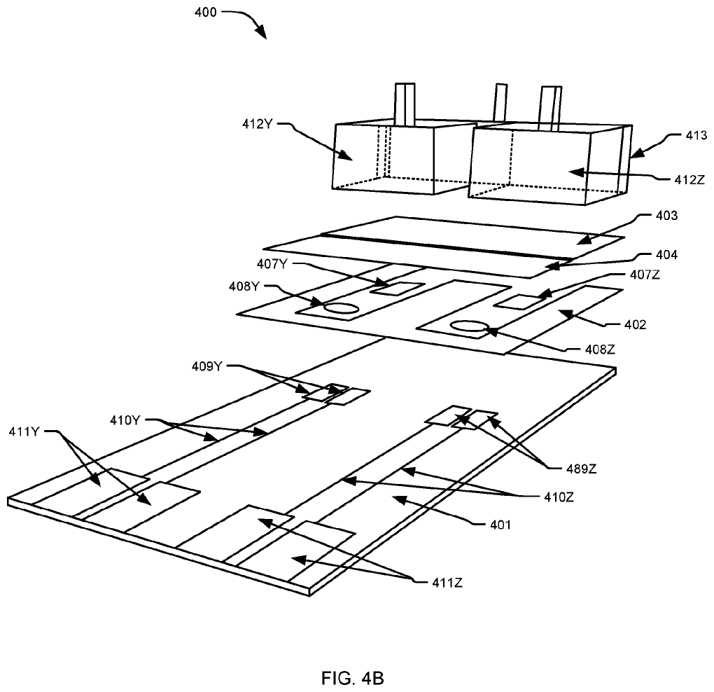

FIG. 4A illustrates top view of a fourth embodiment of the proposed biosensor having independent processing in each channel, in accordance with an embodiment of the present disclosure.

FIG. 4B illustrates exploded view of the fourth embodiment of the proposed biosensor with pressers, in accordance with an embodiment of the present disclosure.

As illustrated in FIGS. 4A and 4B, a fourth embodiment of the proposed biosensor 400 is disclosed. The biosensor 400 can include independent multiple containment chambers and channels to simultaneously determine the concentration of two or more analytes based on the number of channels provided on the substrate.

The creatine present in blood can act as positive interferent. As can be seen in reaction set 1,2,3, the biosensors would measure the total concentration (z) of creatine (y)+creatinine (x). It should be noted that the concentration of the creatine can be in same range as that of creatinine in the case of human blood samples. Thus, it becomes necessary to subtract the concentration of the creatine from the total concentration.

As a result, the fourth embodiment of the proposed biosensor 400 can be provided with two sets of detection and preprocessing reagents to facilitate simultaneous determining of concentration of z (creatinine+creatine) and y (creatine). Further, the electronic device that measures these concentrations, can subtract the later value from the former and can give the true creatinine concentration (x) value present in the fluid.

In an embodiment, the bio sensor 400 can include a substrate 400. The spacer 402 can be PET or Polyester based double-sided adhesive tape. Its special shape as shown in FIG. 4B can be cut out of a sheet of double-sided adhesive with help of dye cutter/laser cutter. When the first plate 403 and the second plate 404 are put in place on the substrate 401, a, total of 4 channels are formed. The first two compartments for measurement of creatine (channels y), and the second compartments for detection of total creatine+creatinine (channel z). Moreover, a single sample inlet port 405 is formed and two stopper 406y, 406z for respective channels are formed.

In an embodiment, the first reagents deposition of 407Y does not contain creatininase but creatinase along with buffers, salts, polymers, surfactants and interference removing agents, whereas the first reagents deposition 407Z can contain both creatinase as well as creatininase along with other reagents.

In an embodiment, the detection reagents deposition 408Y and 408Z on the two channels can be similar, and can include sarcosine oxidase, peroxidase and reduced form of the mediator as per reaction set 3 or sarcosine oxidase and the oxidized form of a mediator as per reaction set 2, along with other components like salts, buffers, polymers, and surfactants.

In an embodiment, the transfer of the fluid from either of the containment compartments can be carried out employing two pressers 412Y and 412Z, which can be actuated independently as well as simultaneously as per requirement. To block the backflow from either of the channels, another presser 413 is used. 412Y,Z and 413 are part of an electronic device (not shown) accompanied by the biosensor.

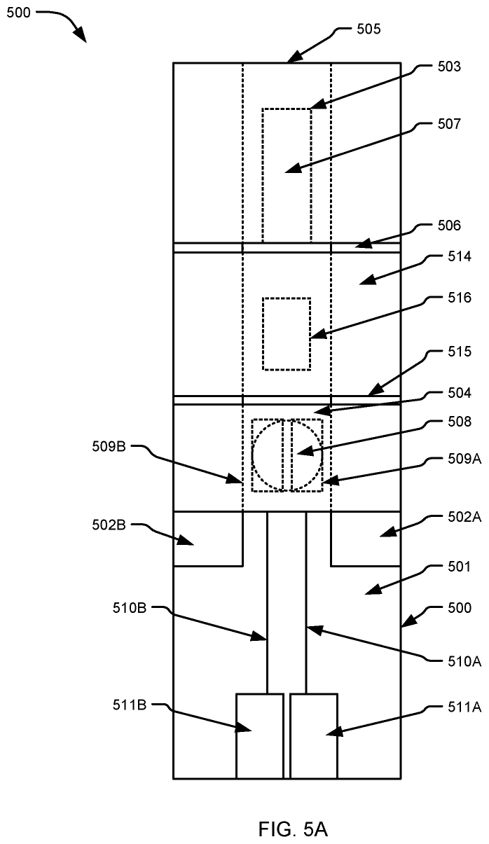

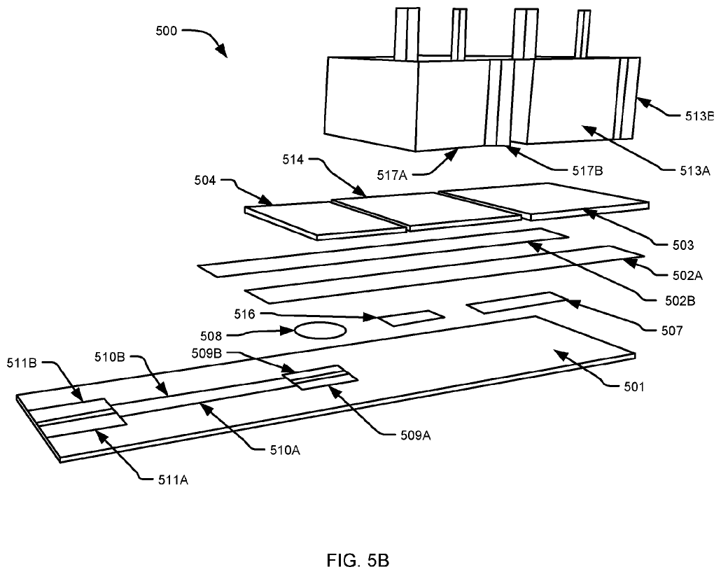

FIG. 5A illustrates top view of a fifth embodiment of the proposed biosensor with multiple containment chambers, in accordance with an embodiment of the present disclosure

FIG. 5B illustrates exploded view of the fifth embodiment of the proposed biosensor with pressers, in accordance with an embodiment of the present disclosure.

As illustrated in FIGS. 5A and 5B, in an embodiment, the proposed biosensor 500 can include an additional containment chamber to further overcome the creatine correction as solved by the fourth embodiment of the present invention.

In an embodiment, the bio sensor 500 can include a third plate 504 after the second plate 514. The third plate 504 can be configured over the substrate to form a third compartment in the channel, between the third plate 504 and the substrate 501. The third containment chamber can be fluidically separated from the second containment chamber by a second predetermined distance.

In an embodiment, the fluid can be configured to flow from the first containment chamber into the second containment chamber only when the first predefined pressure is applied on the first plate. Further, the fluid can be configured to flow from the second containment chamber into the third containment chamber only when a second predefined pressure is applied on the second containment chamber.

In an implementation, the fluid can be inserted into the first containment chamber through an inlet port 505, where creatinase, sarcosine oxidase, and catalase are present in a non-dissolving reagent layer 507. The fluid can encounter a discontinuity 506 between the first containment chamber and the second containment chamber, and does not fill the biosensor further. The first containment chamber can optionally and more advantageously contain interference removing agents. As a result, all the creatine present in fluid is degraded in this way. The first presser set 513A,B can then enable the transfer of the fluid from the first containment chamber to the second containment chamber.

To make sure that fluid in the first containment chamber does not flow out of the inlet port 505, two pressers 513A and 513B are involved. The presser 513B can have very less area as compared to the presser 513A, and which presses the first plate 503 at a point closer to inlet port, in such a way that when after this, the presser 513A is pressed, it does not allow backflow of the fluid, and the fluid is transferred in a single direction i.e. towards the second containment chamber.

In an embodiment, the second containment chamber can include the detection reagents 516 including creatininase, creatinase and interference killing agents, and the likes. Thus, only creatinine (not creatine) present in the fluid can survive as sarcosine is in this second containment chamber.

In an implementation, after sufficient time in the second containment chamber, a second presser set 517 enables the transfer of the fluid from the second containment chamber to the third containment chamber for detection. To make sure that the fluid in the second containment chamber does not recede into the first containment chamber, two pressers 517A and 517B are involved. The presser 517B can have a very less area as compared to the presser 517A, and which presses the third plate 514 at a point closer to the first containment chamber, in such a way that when after this, the presser 517A is pressed, it does not allow backflow of the fluid, and the fluid is transferred in a single direction i.e. towards the third containment chamber.

In an embodiment, the third containment chamber can be deposited with detection reagents formulation 508 for the detection of sarcosine produced in the second containment chambers. The detection reagent 508 can contain sarcosine oxidase, peroxidase and reduced form of mediator along with buffers, salts, polymers, and surfactants. Alternatively, reagent formulation 508 can contain sarcosine oxidase along with the oxidized form of mediator along with other film-forming reagents. In an exemplary embodiment, the third containment chamber can also include electrodes 509A,B connected to pads 511A,B through connectors 510A,B.

In an implementation, the first containment chamber can contain Ferricyanide which can react with such components of the fluid that can disturb the final results during detection, as a result many other interference removal agents are not required in the first containment chamber. Further, the second containment chamber can contain an agent that can reduce excess Ferricyanide along with creatinase and creatininase, fluid being received from the first containment chamber. These agents preferably do not dissolve in the fluid and also do preferably not reduce other components which were oxidised by ferricyanide. In an exemplary embodiment, these agents can be FAD dependent glucose dehydrogenase which can utilize glucose present in the fluid and react with remaining Ferricyanide to produce ferrocyanide. Furthermore, the fluid can be transferred to the third containment chamber for final detection of the analytes. However, the choice of enzyme is not limited to glucose dehydrogenase. Any other dehydrogenase can be used as such which is reactive to ferricyanide.

In an implementation, the first containment chamber might contain any oxidising agent including an elecrto-chemical mediator or hydrogen peroxide generating reagent that reacts with such components of the fluid that can disturb the final results during detection, as a result many other interference removal agents are not required in the first containment chamber.

It should, however, be noted that this invention is not limited to amperometric detection of creatinine in blood. The example of amperometric detection of creatinine serves as a good benchmark for various applications of the present invention like dissolved oxygen interference removal, metabolite interference removal, and detection channel reagent load reduction. The present invention can be used with modified detection reagent and pre-processing reagent layers to conduct interference-free detection of various blood analytes including glucose, cholesterol, triglycerides, lipoproteins, other blood protein markers like HbA1C amongst others. The present invention should be understood in terms of ability to contain a sample on-chip for a desired amount of time (or a series of containment chamber and can be transferred in a controlled manner to next containment chamber for preprocessing and/or detection) where a fluid sample can be modified in any desired manner without interfering with the main detection system.

While the foregoing describes various embodiments of the invention, other and further embodiments of the invention may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

### Advantages of the Invention

The proposed invention provides a biosensor and a method for detecting one or more analytes present in a fluid.

The proposed invention processes and detects analytes present in a fluid in multiple steps on a single biosensor.

The proposed invention mitigates the effects of interferents and reducing the load of reagents on the channels of a biosensor.

The proposed invention immobilizes incompatible reagents on the biosensor.

The proposed invention detects multiple analytes with same amount of fluid.

The proposed invention provides a biosensor allowing simpler calibration.

The proposed invention provides a biosensor having improved reagent load distribution.

The proposed invention detects analytes present in whole blood where the detection of analytes is subjected to a lot of non-specific interferences.

The proposed invention provides a method to detect creatine, creatinine, and other analytes present in whole blood, mitigating the effects of the interferents.

The proposed invention removes interferents from sample fluid prior to detection of the analytes of the bio sensor.

The proposed invention reduces load of reagents on the channels (and containment chambers) of the biosensor, for faster and enhanced processing and detection of the analytes.

I claim:

1. A sensor for detection of analyte in a fluid, the sensor comprising:
a substrate;
a first plate configured on the substrate to form at least one channel between the first plate and the substrate, each of the at least one channel comprises a first containment chamber configured to receive and get filled with the fluid,
a second plate positioned at a first predetermined distance from the first plate, and configured on the substrate to form a second containment chamber in each of the at least one channel,
wherein the containment chamber and the second containment chamber of each of the at least one channel are fluidically separated from each other such that the fluid is configured to flow between the corresponding containment chambers only when a predefined pressure is applied on the corresponding plate; and
wherein the each of the at least one channel are mechanically separated from each other; and
wherein the first containment chamber and the second containment chamber associated with each of the at least one channel facilitates conversion of one or more analytes present in the fluid into one or more detectable compounds;
wherein the sensor is configured to be operatively coupled to a detection device selected from a group comprising optical device, electrochemical device, electrostatic device, piezoelectric device, electromagnetic device, and any or a combination of a thermal sensor, and a mass sensor.

2. The sensor as claimed in claim 1, wherein the sensor comprises at least one spacer configured between the substrate, and the first plate and the second plate to form the first containment chamber and the second containment chamber respectively.

3. The sensor as claimed in claim 3, wherein the at least one spacer comprises:
a first set of spacers having a first predefined thickness, and positioned between the first plate and the substrate to form the corresponding first containment chamber of the at least one channel, having the first predefined thickness; and
a second set of spacers having a second predefined thickness, and positioned between the second plate and the substrate to form the corresponding second containment chamber of the at least one channel, having the second predefined thickness.

4. The sensor as claimed in claim 1, wherein the sensor is adapted to be configured with at least one presser, and wherein the at least one presser is configured to facilitate application of pressure on at least one plate to enable flow of the fluid between the corresponding containment chambers.

5. The sensor as claimed in claim 1, wherein the sensor comprises at least one third plate positioned at a second predefined distance from the second plate, and configured over the substrate to form a third containment chamber in each of the at least one channel.

6. The sensor as claimed in claim 5, wherein the third containment chamber are fluidically separated from the corresponding second containment chamber such that that the fluid is configured to flow between the third containment chamber and the second containment chamber only when the predefined pressure is applied on at least one of the corresponding plates.

7. The sensor as claimed in claim 1, wherein the first containment chamber and the second containment chamber comprises any or a combination of a first set of reagents and a second set of reagents respectively to facilitate any or a combination of processing of the fluid, and conversion of one or more analytes present in the fluid into one or more detectable compounds, and wherein the first set of reagents comprises one or more interference removing reagents adapted to remove one or more interferents from the fluid.

8. The sensor as claimed in claim 7, wherein the one or more interferents comprises Absorbic acid, and wherein the one or more interference removing reagents comprises Ascorbate Oxidase, Ascorbic Acid Oxidase, Glucose Oxidase, and oxidized from of the mediators.

9. The sensor as claimed in claim 1, wherein the one or more analytes comprises any or a combination of glucose, cholesterol, HbA1C, protein, and bilirubin, and wherein the one or more interferent comprises any or a combination of bilirubin, oxygen, glucosem, proteins, urea, and uric acid, and wherein the one or more interference removing reagents comprises any or a combination of oxidizing agents, reducing agents.

10. The sensor as claimed in claim 1, wherein one of the containment chamber comprises a first agent that reacts with at least one component present in the fluid or produces a second agent that reacts with at least one component present in the fluid, and wherein another containment chamber comprises a third agent that removes any or a combination of the first agent and the second agent from the fluid.

11. The sensor as claimed in claim 10, wherein the first agent second agent and the third agent are selected from a group comprising ferricyanide, ferrocyanide, hydrogen peroxide, oxygen, an oxidising agent, a reducing agent, an electrochemical mediator, an enzyme, glucose, urea, and uric acid.

12. The sensor as claimed in claim 10, wherein the first agent is ferricyanide, and the third agent is glucose dehydrogenase.

13. The sensor as claimed in claim 1, wherein one of the containment chambers corresponding to a first channel among the at least one channel comprises a predetermined quantity of the analyte or its derivative, wherein measurement for the analyte in the fluid and measurement for the analyte in the fluid enriched with derivative is made.

14. The sensor as claimed in claim 13, wherein more than one of the containment chambers associated with one of the channels among the at least one channel allows detection of a different analyte.

15. A method for detection of an analyte in a biological fluid, the method comprising:
retaining, at a first spatial region, the biological fluid for a predefined period of time, wherein the first spatial region allows a first set of reactions to be performed on the biological fluid; and
transferring, the biological fluid from the first spatial region to a second spatial region, wherein movement of the fluid from the first spatial region to the second spatial region is controlled,
converting, at a second spatial location, the analytes present in the biological fluid into a final detectable entity.

16. The method as claimed in claim 15, wherein the first set of reaction comprises creatinine conversion to creatine and creatine conversion to sarcosine.

17. A sensor for detection of at least one analyte in a fluid, wherein the sensor comprises:
at least one channel, wherein at least one of the channels comprises at least one stopper in order to contain the fluid such that at least one containment chamber is formed in each of the at least one channel; and
wherein, the at least one of the containment chambers comprises any or a combination of fluid processsing, analyte detection, sample pretreatment, interference removal, analyte enrichment, dissolved oxygen removal, dissolved oxygen enrichment, analyte derivative enrichment, partial reaction completion; and
wherein the at least one stopper comprises any or a combination of capillary break, a hydrophobic coating, a discontinuity in hydrophillic coating, and wherein transfer of sample between consecutive containment chamber separated by stopper is controlled externally by a device provided with the biosensor,

18. The sensor as claimed in claim 17, wherein one of the containment chamber comprises a first agent that reacts with at least one component present in the fluid to produces a second agent that reacts with at least one component present in the fluid, and wherein another containment chamber comprises a third agent that removes any or a combination of the first agent and the second agent from the fluid.

19. The sensor as claimed in claim 18, wherein the first agent second agent and the third agent are selected from a group comprising ferricyanide, ferrocyanide, hydrogen peroxide, oxygen, an oxidising agent, a reducing agent, an electrochemical mediator, an enzyme, glucose, urea, and uric acid.

20. The sensor as claimed in claim 18, wherein the first agent is ferricyanide, and the third agent is glucose dehydrogenase.

21. The sensor as claimed in claim 17, wherein one of the containment chambers corresponding to a first channel among the at least one channel comprises a predetermined quantity of the analyte or its derivative, wherein measurement for the analyte in the fluid and measurement for the analyte in the fluid enriched with derivative is made.

22. The sensor as claimed in claim 17, wherein more than one of the containment chambers associated with one of the channels among the at least one channel allows detection of a different analyte.
